(19) United States
(12) Patent Application Publication
(10) Pub. No.: US 2005/0049652 A1
(43) Pub. Date: Mar. 3, 2005

(54) Functional electrical stimulation system

(76) Inventor: Kai-yu Tong, Hong Kong (HK)

Correspondence Address: ALIX YALE & RISTAS LLP, 750 MAIN STREET, SUITE 1400, HARTFORD, CT, 06103, US

(21) Appl. No.: 10/647,488

(22) Filed: Aug. 25, 2003

(51) Int. Cl. A61N 1/18

(52) U.S. Cl. 607/48; 607/49; 607/62

(57) ABSTRACT

An electrical stimulation device has a sensor for detecting a movement event of a body part and an electrode for making electrical contact with an area of the body part. A controller coupled to the sensor and electrode receives a sensor signal indicating the movement event, and outputs to the electrode an output comprising a rise signal, a stimulation signal and a fall signal. The computer records a duration of use and a number of movement events during the duration of use in a log file. A handheld remote control allows stimulation data to be sent to the device and stored in a stimulation file.

### BACKGROUND TO THE INVENTION

1. Field of the Invention

The present invention relates to Functional Electrical Stimulation Systems.

2. Background Information

Functional Electrical Stimulation (FES) systems artificially stimulate the muscles, and muscle groups, of persons through the use of electrical current in order to stimulate movement. As early as 1971 Liberson applied electrical stimulation to assist walking in patients with foot drop.

Most Functional Electrical Stimulation systems are designed for rehabilitation of spinal core insured or stroke patients. There are a number of FES systems on the market to assist foot drop in persons who have had a stroke. Typically, a heel switch is provided on the foot of a patient to indicate when the patient lifts their foot off the ground so that a controller can stimulate appropriate muscles to contract to raise the foot during the step.

Current systems suffer two main drawbacks. Firstly, it is not convenient to tune the electrical stimulation pulse characteristics to suit different patients during treatment because known apparatus do not have external controls to avoid unintentional tuning by patients. Secondly, known devices do not provide any information on a patient's adaptability to the device and improvement in step performance during the treatment.

### SUMMARY OF THE INVENTION

It is an object of the present invention to provide a Functional Electrical Stimulation systems which assists with rehabilitation of stroke patients, or which at least offers a useful choice.

According to a first aspect of the invention there is provided an electrical stimulation device comprising: a sensor for detecting a movement event of a body part, an electrode for making electrical contact with an area of the body part, and a controller coupled to the sensor and electrode for receiving a sensor signal indicating the movement event, and for outputting to the electrode an output comprising a rise signal, a stimulation signal and a fall signal, and programmed to record a duration of use and a number of movement events during the duration of use.

According to a second aspect of the invention there is provided an electrical stimulation device for controlling the movement of a body part comprising: a sensor for detecting a movement event of a body part, an electrode for making electrical contact with an area of the body part and for stimulating a muscle of the body part, a housing to be worn by a user of the device, receiver on the housing for receiving wireless signals from a remote unit, and a controller provided in the housing and coupled to the receiver for receiving stimulation data from the remote unit and storing the stimulation data in a stimulation file, and coupled to the sensor for receiving a sensor signal indicating the movement event, and for generating a control signal using the stimulation file in response to the movement event, and for outputting the control signal to the electrode.

Preferably, the controller is also programmed for generating a log file storing a duration of use and a number of movement events during the duration.

Preferably, the stimulation data includes a stimulation intensity level, a rise time, a stimulation time, and a fall time.

Preferably, the stimulation data also includes a pulse form, a triggering period, a triggering method and triggering criteria.

Preferably, the device further includes a computer removably coupled to the controller for downloading the stimulation file and log file, and for updating the stimulation file, and programmed to store data from the stimulation and log files in a database, and for outputting for display the stimulation data and the duration of use and the number of movement events.

Preferably, the computer is a Personal Digital Assistant.

Preferably, the database also includes information about the user of the device, and the computer is programmed for accessing the database by a Windows™ graphical user interface.

Preferably, the remote unit is a handheld remote control unit.

Preferably, the body part is a foot and the sensor is a heel switch.

Further aspects of the invention will become apparent from the following description, which is given by way of example only.

### BRIEF DESCRIPTION OF THE DRAWINGS

Embodiments of the invention will now be described with reference to the accompanying drawings in which:

FIG. 1 is a schematic of the leg of a person with a Functional Electrical Stimulation (FES) device fitted,

FIG. 2 is an exploded front and back view of a wearable control module for the FES device,

FIG. 3 is an electrical schematic for the control module,

FIG. 4 is a Graphical User Interface (GUI) for displaying and setting simulation data on a personal computer,

FIG. 5 is a relationship diagram of database records for use in a Functional Electrical Stimulation rehabilitation system,

FIGS. 6 to 28 illustrate a Window™ GUI for assessing and updating the FES systems database,

FIG. 29 is a perspective view of a Personal Digital Assistant (PDA) for interfacing with the FES system, and

FIG. 30 is a handheld remote control unit for setting and updating simulation data of the FES device.

### DESCRIPTION OF THE PREFERRED EMBODIMENTS

The preferred embodiment of the invention is an electrical stimulator device to assist in overcoming “foot drop” affecting a person who has had a stroke. A heel pressure switch is used to control the application of electrical pulses which, in turn, stimulate appropriate muscles to contract to raise the foot during step.

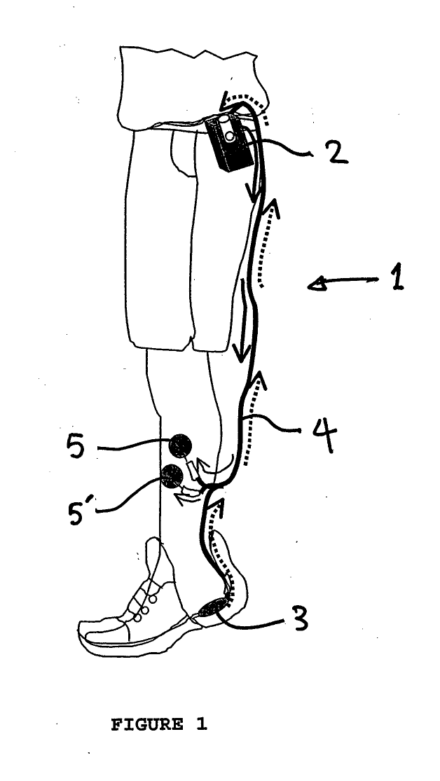

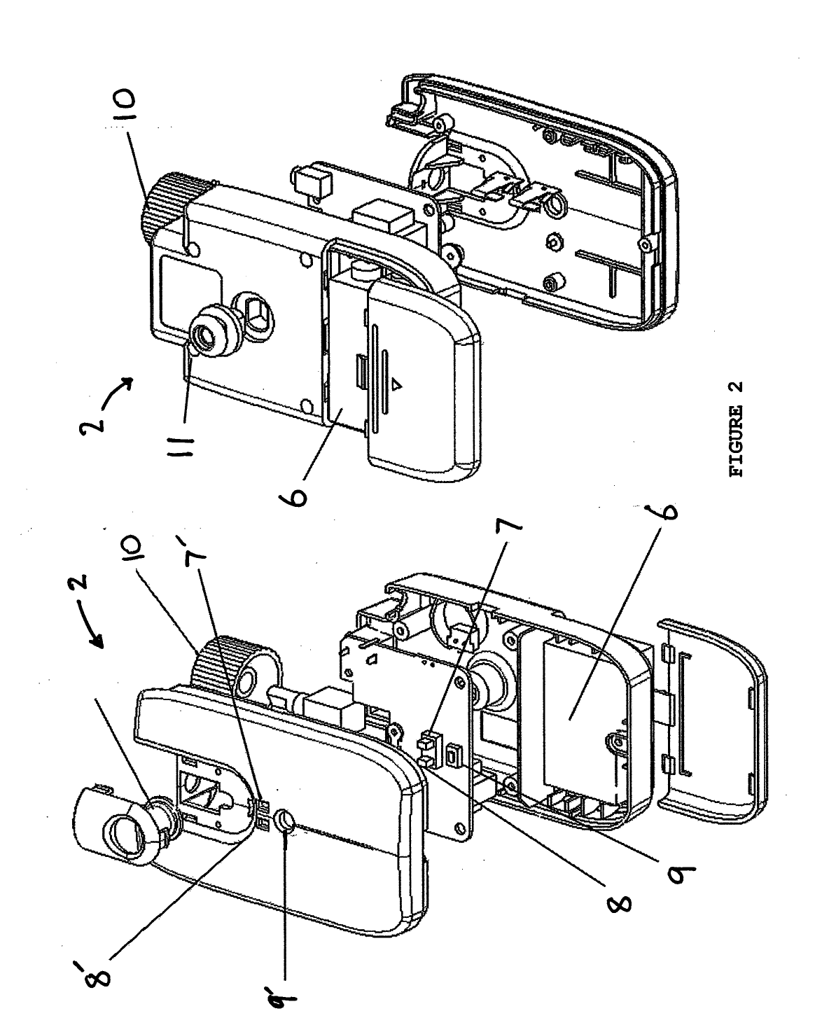

Referring to FIGS. 1 and 2, an electrical stimulation device for assisting with “foot drop” in a person includes a control module 2 which can be worn on the belt of a patient. An input sensor in the form of heel switch 3 is provided to detect when the patient lifts their foot off the ground. Heel switch 3 is connected to control module 2 by wires 4. Electrodes 5 are positioned on the front part of the patient's leg and connected to control module 2 via wiring 4. The control module 2 includes a micro controller which is coupled to sensor 3 and electrode 5.

The control module 2 worn by a user includes a housing containing a micro controller for receiving an event signal from the heel switch 3 indicating that the user has lifted their foot. The controller generates a simulation signal and outputs it to the electrode 5 to simulate muscles which contract to lift the foot during the step. The housing includes a battery 6 for powering the controller. On a front portion of the housing are a battery light emitting diode 7 and a simulation light emitting diode 8 to indicate when a simulation is occurring. A test button 9 is provided for simulating the input from the heel switch 3 to cause a simulation output to the electrode 5. On one end of the housing is a rotary knob 10 for turning the module 2 on/off and adjusting the simulation intensity level. On the back of the housing is a belt clip adaptor 11 so that the housing can be worn by the user of the device.

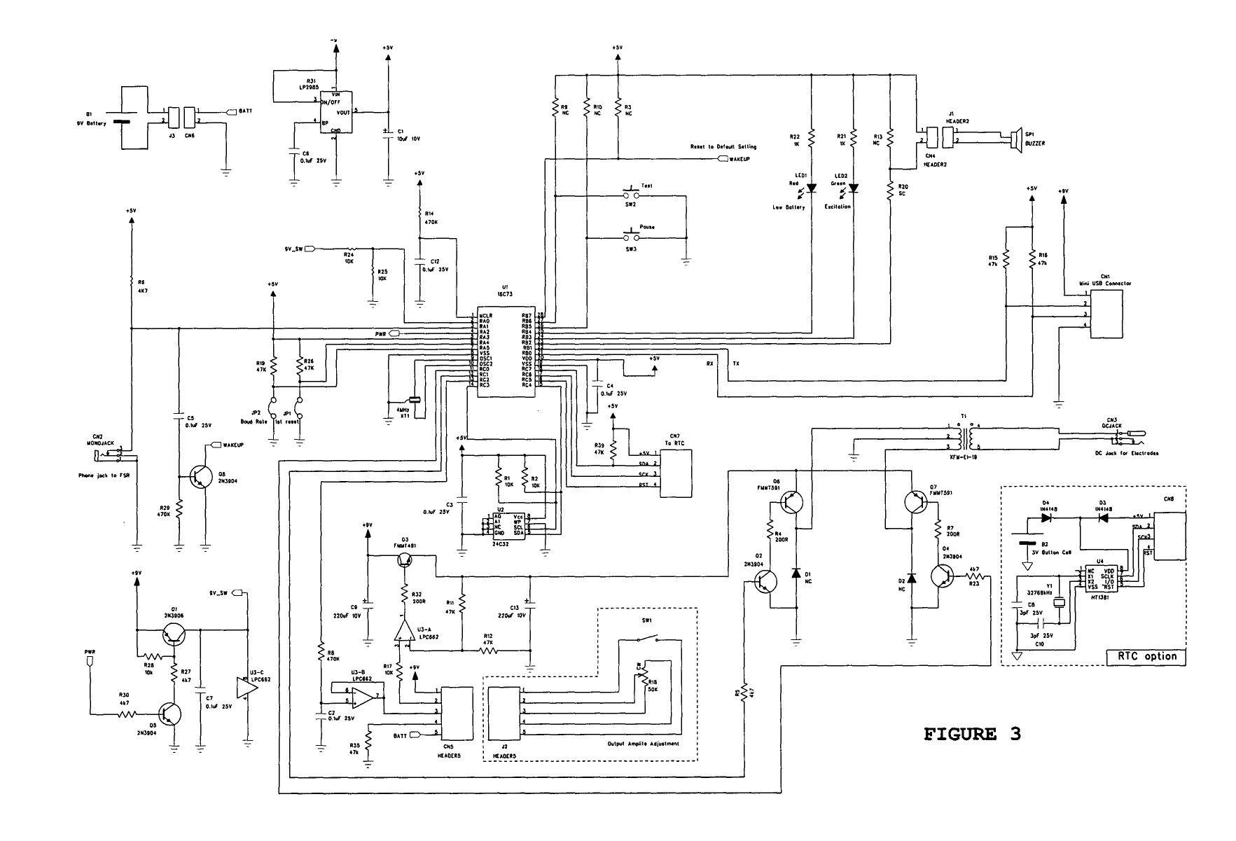

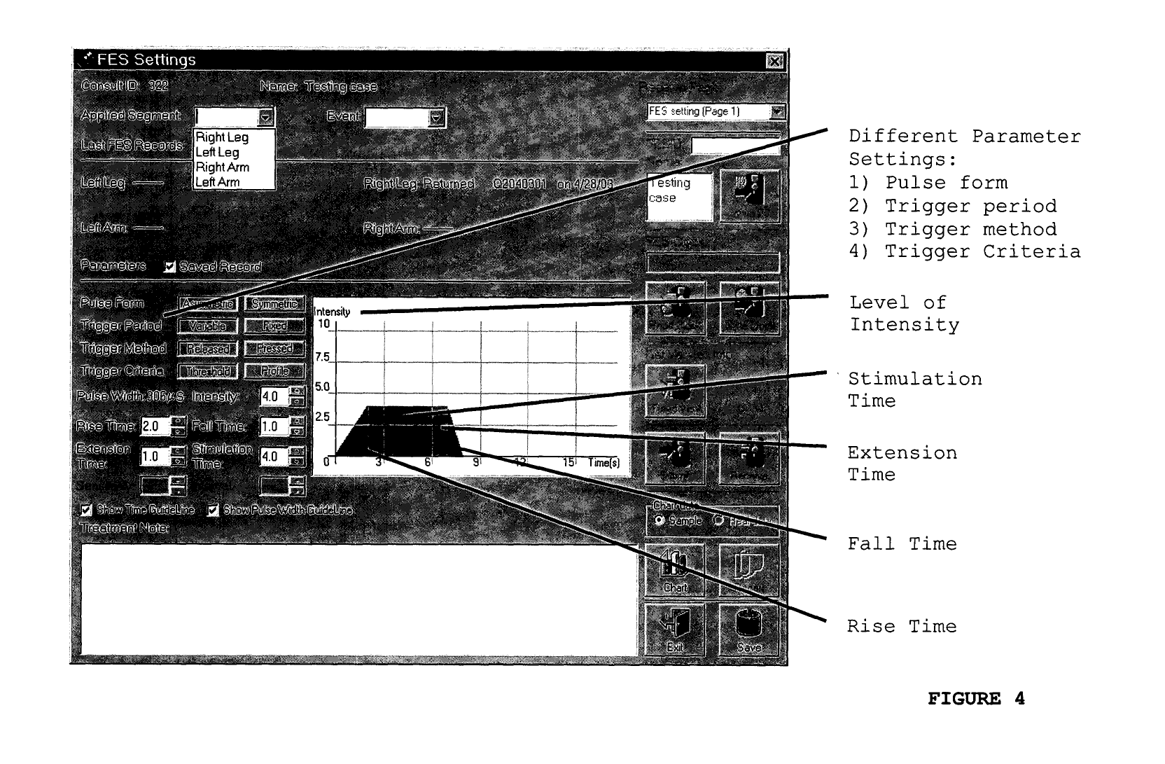

Referring to FIG. 3, a schematic diagram of the controller and associated electrical circuit is shown.

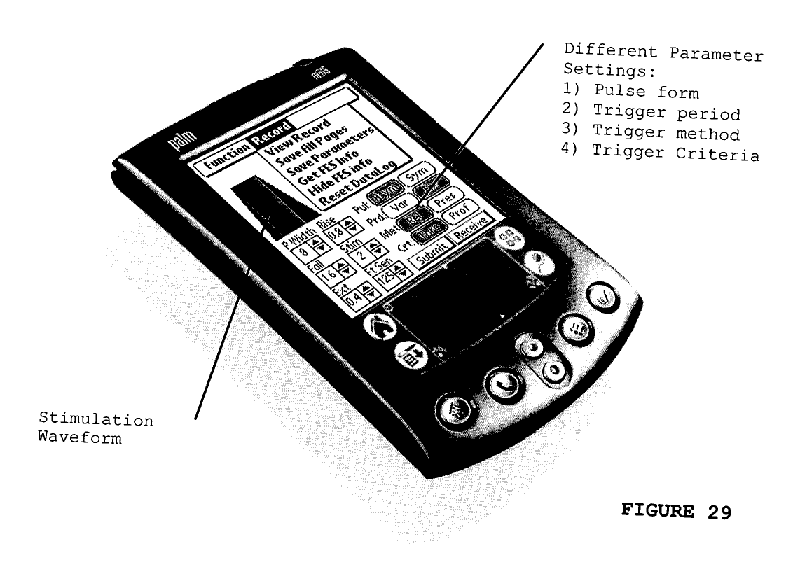

The computer also has a data logging function to record the hours of use and number of steps walked by the patient. This information is recorded in a log file which can be downloaded to a computer database or Personal Digital Assistant (PDA), shown in FIG. 29. The information can be displayed on the computer of PDA screen.

By logging duration of use and number of steps taken the physician is able to know more about the daily life pattern of patients and so can help prescribe a more suitable time schedule of walking exercise for each individual user. The logged information can also be used to monitor overuse or insufficient walking exercise by patients. Patients can adapt to the device more effectively so that they can improve their quality of life by merging into the society again.

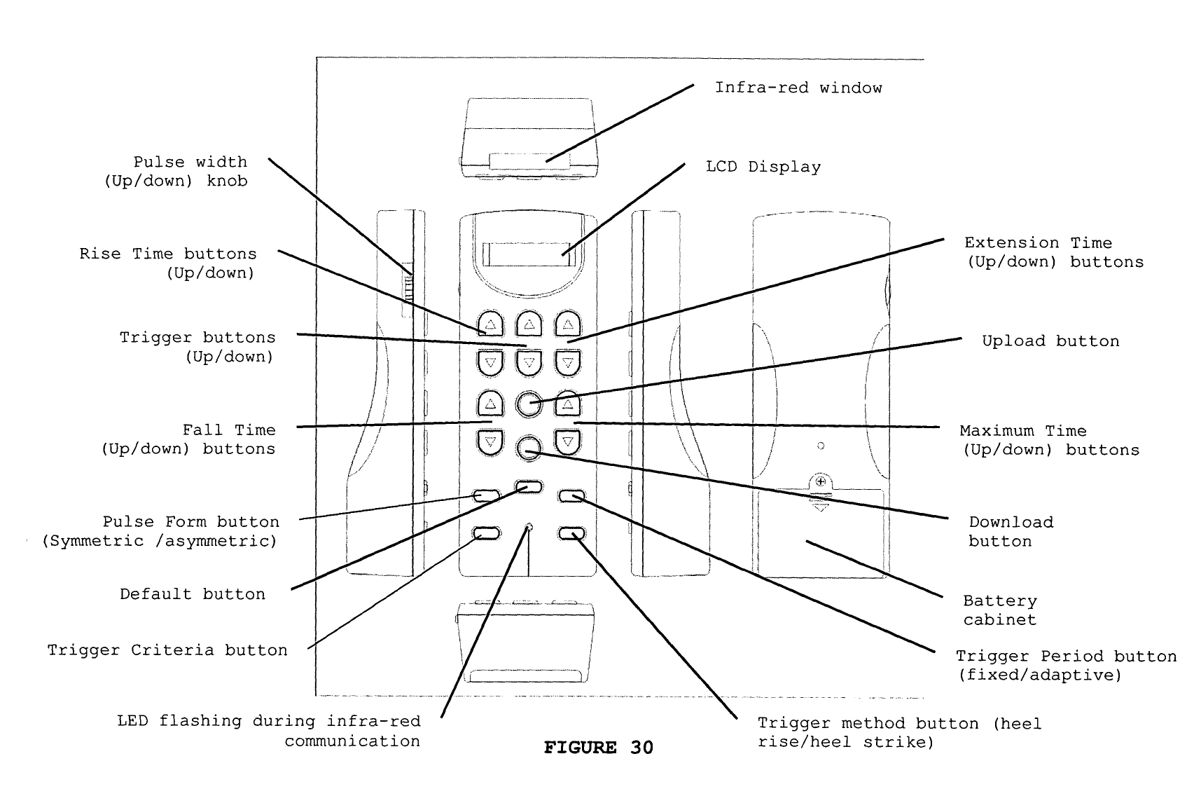

The housing also has a wireless communications receiver, such as an infra-red receiver, for receiving stimulation data from a remote handheld control unit, shown in FIG. 30. The remote control allows a physician to set stimulation parameters to fit individual patients. The stimulation parameter data is sent to the controller and stored in a stimulation file which the controller uses to generate the stimulation signal when a step event is recorded by the heel switch. The wireless remote control makes stimulation parameter setting more effective and efficient. The physician can watch a patient and evaluate their walking characteristics while using the device. Immediate changes to the stimulation parameters can be made and the results observed without stopping the patient. As a consequence, patients will find it easier to adapt to the device and physicians will take less time in adjusting the device's parameters to fit different patients.

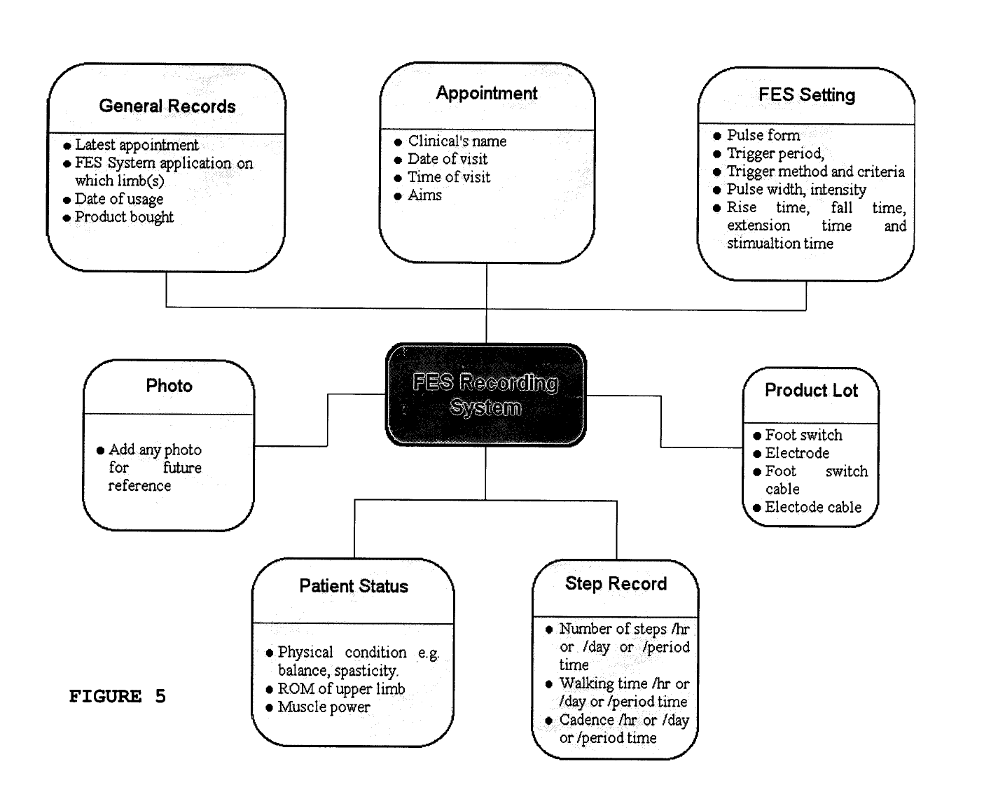

The computer and PDA software allows logged data to be retrieved from the device and recorded in a database for immediate or later analysis, and displayed on a screen. The PC software design is based on the familiar and user-friendly Windows™ Graphical User Interface. The Database contains a full range of patient data, as indicated in FIG. 5, to allow the physician to retrieve and save all the patient records, which include all the parameters stored inside the stimulation devices.

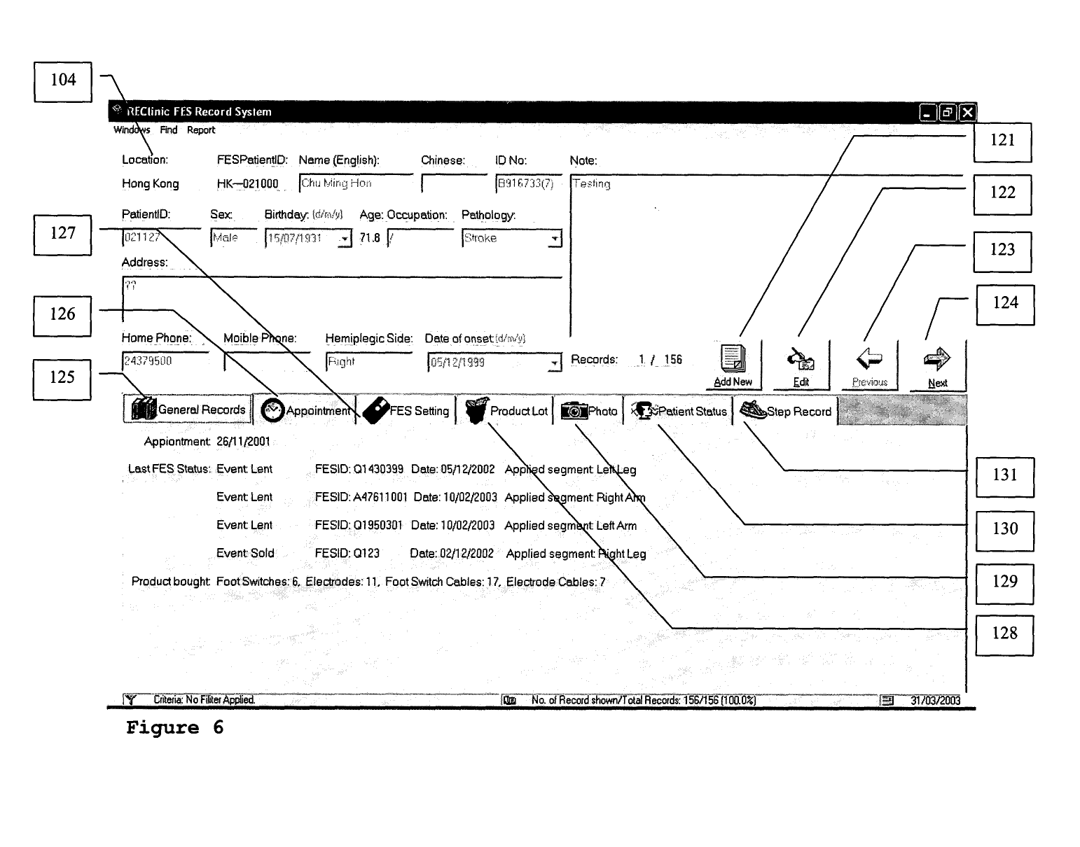

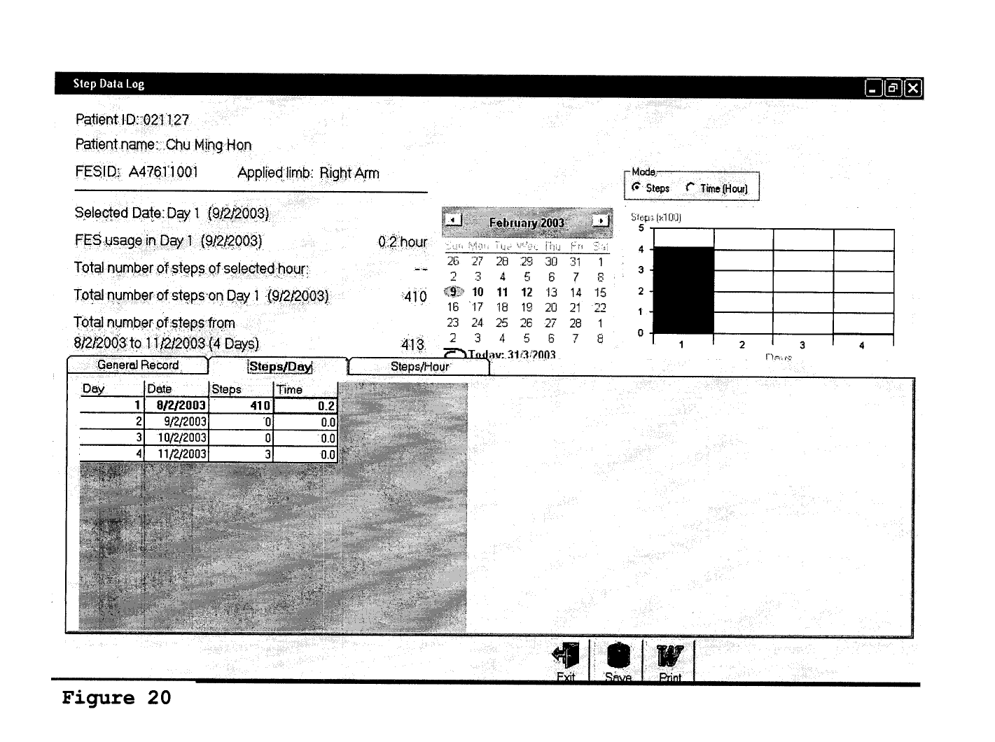

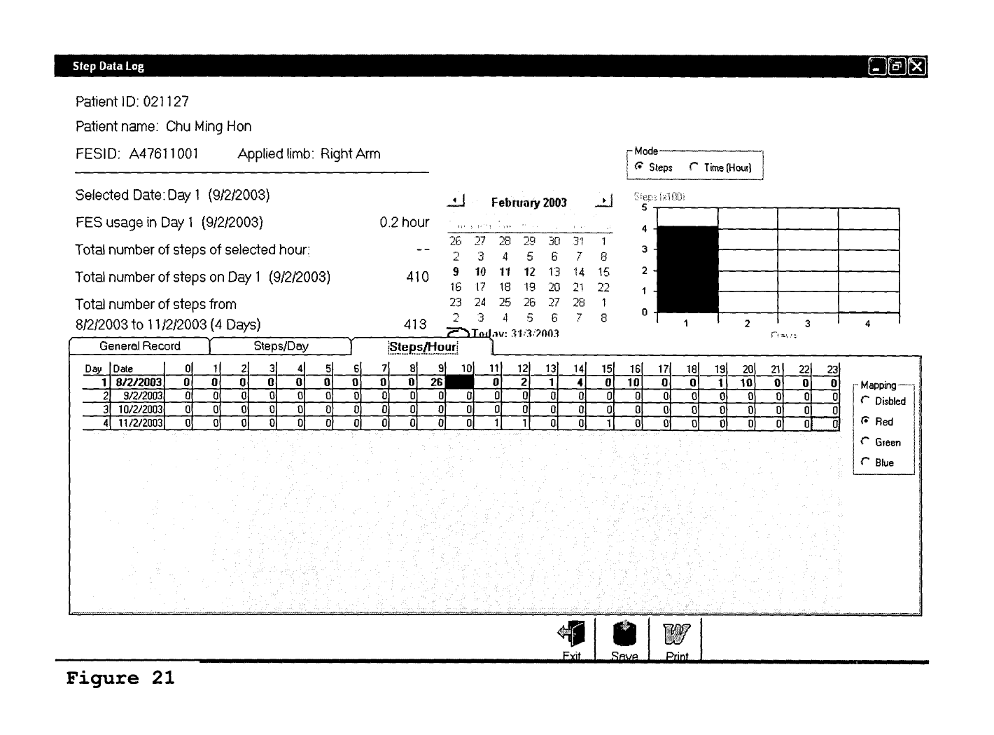

Referring to FIGS. 6 to 28, Functional Electrical System (FES) Recording System comprises the computer database and Windows GUI to allow storage of patient details and FES system parameters, and to allow monitoring patient's mobility as treatment progress.

FIG. 6 illustrates the main window of the FES record system. The upper half of the window shows patient information including, for example, the patient's name, diagnosis, and address. The lower half of the window can be used to show other patient information arranged under seven pages accessible by tabs 125, 126, 127, 128, 129 130 and 131.

General Records 125 summaries important record information including appointment status, whether the patient is using the FES system or not and FES accessories bought so far. It provides a fast overview of the patient's information.

Appointment 126 shows information of appointments so that clinician can check progress of treatment.

FES Setting 127 shows the setting of the FES system during each patient visit to allow a continuous service to the patient by tracking the effect of different FES settings on the patient.

Product Lot 128 shows the date and number of accessories the patient has bought.

Photo 129 allows the clinician to upload digital images into the FES Recording System to supplement the patient's records visually. For example, an image may show the patient's leg with electrodes attached to show electrode attachment site.

Patient Status 130 shows the patient's physical information such as ROM, walking gait characteristics and muscle power.

Step Record 131 shows the walking pattern of the patient when using the FES System. The record includes the total number of steps, walking time (hours) and cadence per hour, per day or in the whole period.

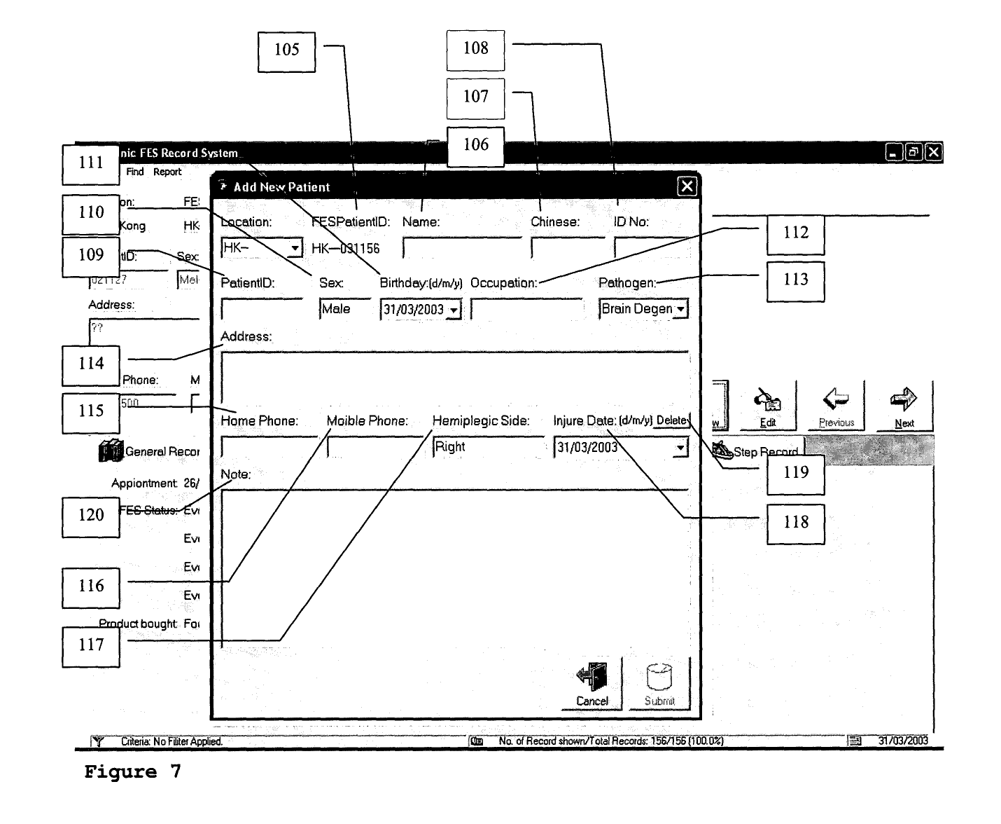

FIG. 7 shows a pop-up window used to add a new patient record. The pop-up window is revealed by clicking “Add New” 121 in the main window. The new patient information is added in each field 106 to 120. In order to saving time patient “Sex” 110 is preset to “Male” and “Hemiplegic Side” 117 is preset to “Right”. To change the former to “Female” or the latter to “Left” or “Bilateral”, click on arrow of the drop-down box.

For the box of “Injure Date” (118), you can input the date of injury in the form of “day/month/year” (d/m/y) or “month/year” (m/y), for those who forget the exact day. For patients who forget the date of injury, you can just choose delete (119) to not input this field. “Note” (120) is the field allow physician to record patient's history other than item provided.

You can edit a patient's information by click the icon “Edit” (122).

Patient records can be reviewed one by one by clicking “Previous” (123) or “Next” (124). To find a patient by name or patient ID, see “Search” screen below.

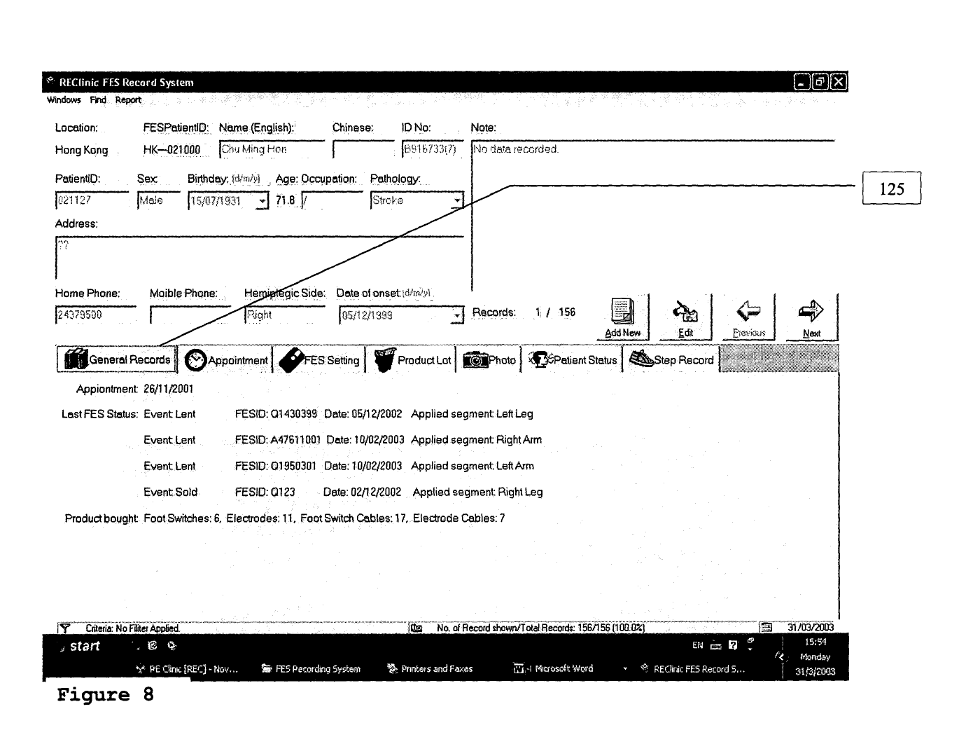

Referring to FIG. 8, general records includes a patient latest appointment, use of the FES System or not, FESID number, the injured body segment, and accessories bought.

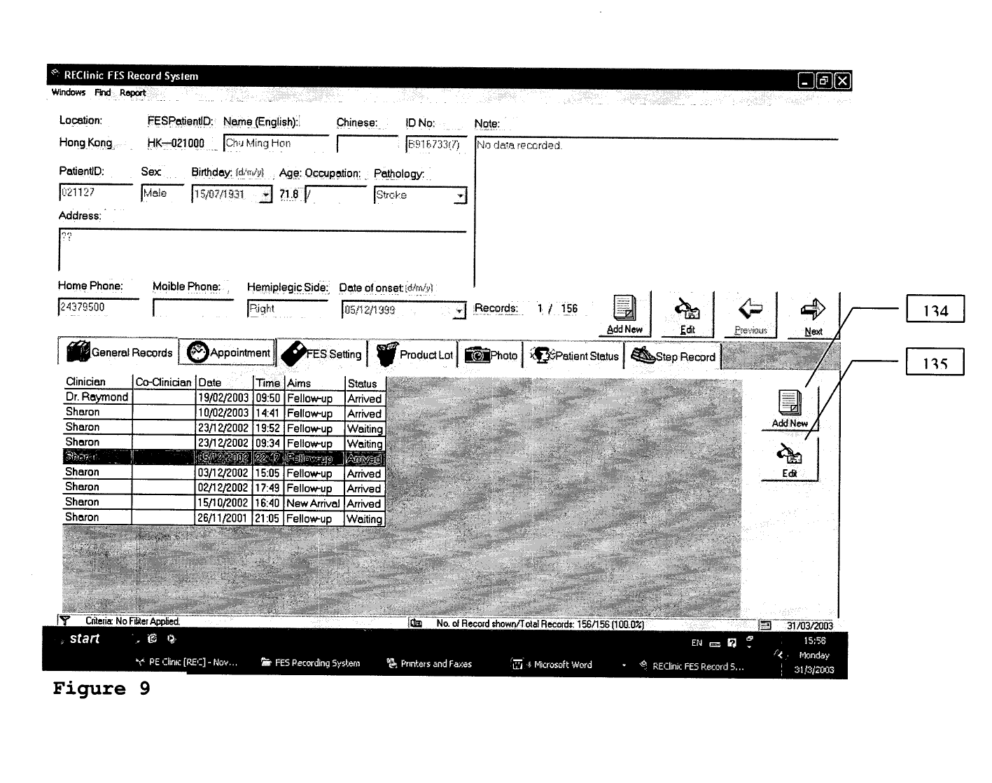

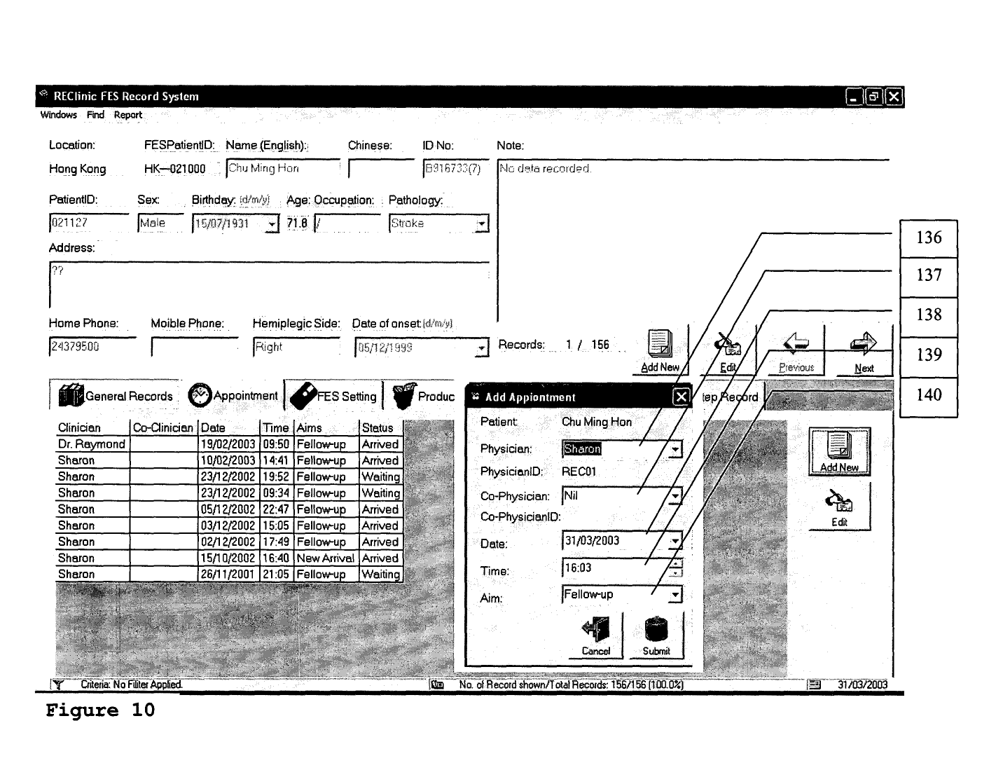

Referring to FIGS. 9 and 10, the FES Recording System allows easy and clear appointment booking. Click the “Add New” (134) and then select physician name (136, 137), date (138) and time of the appointment (139). Also the aim (140) for the appointment, whether it is a first visit or follow up case, etc. Click “Edit” (135) to change an appointment.

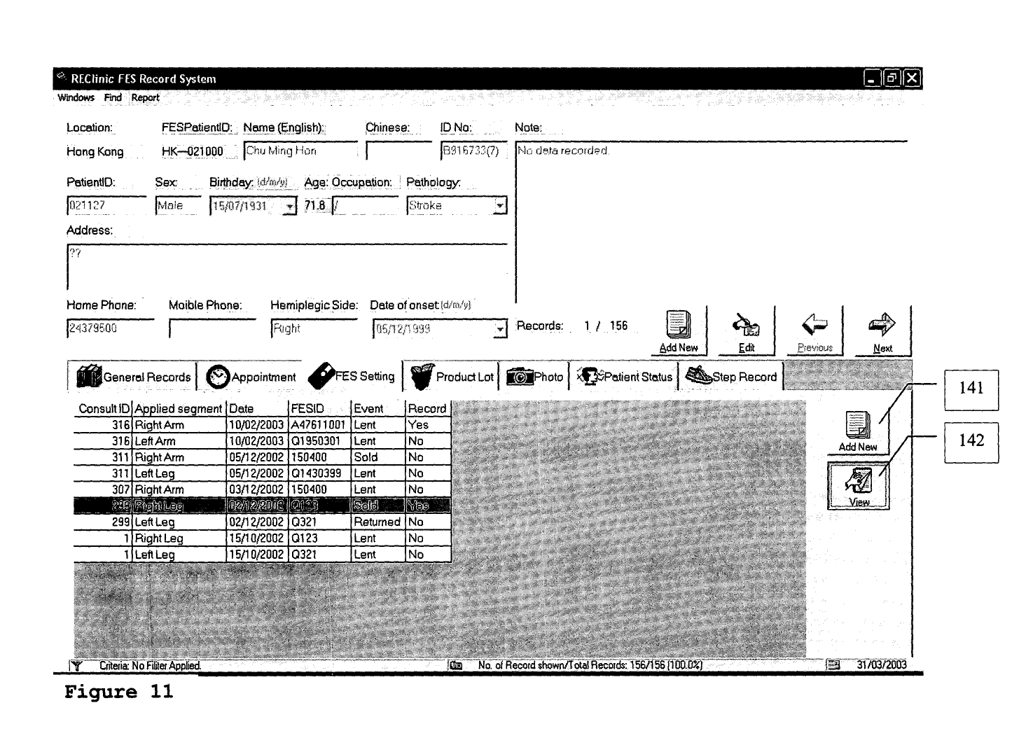

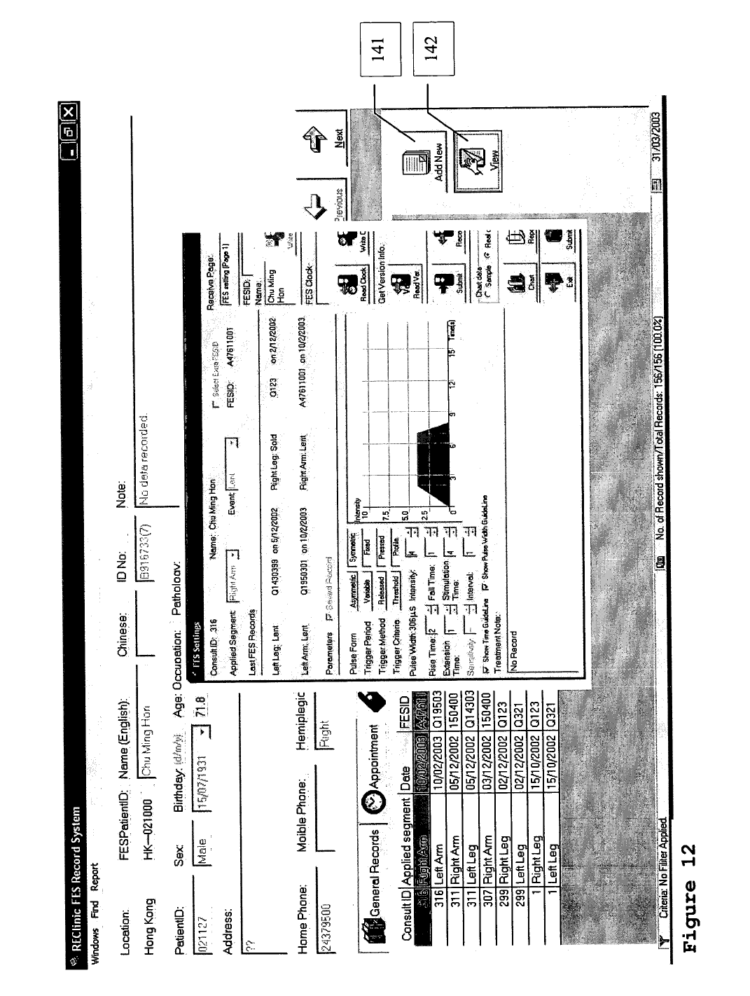

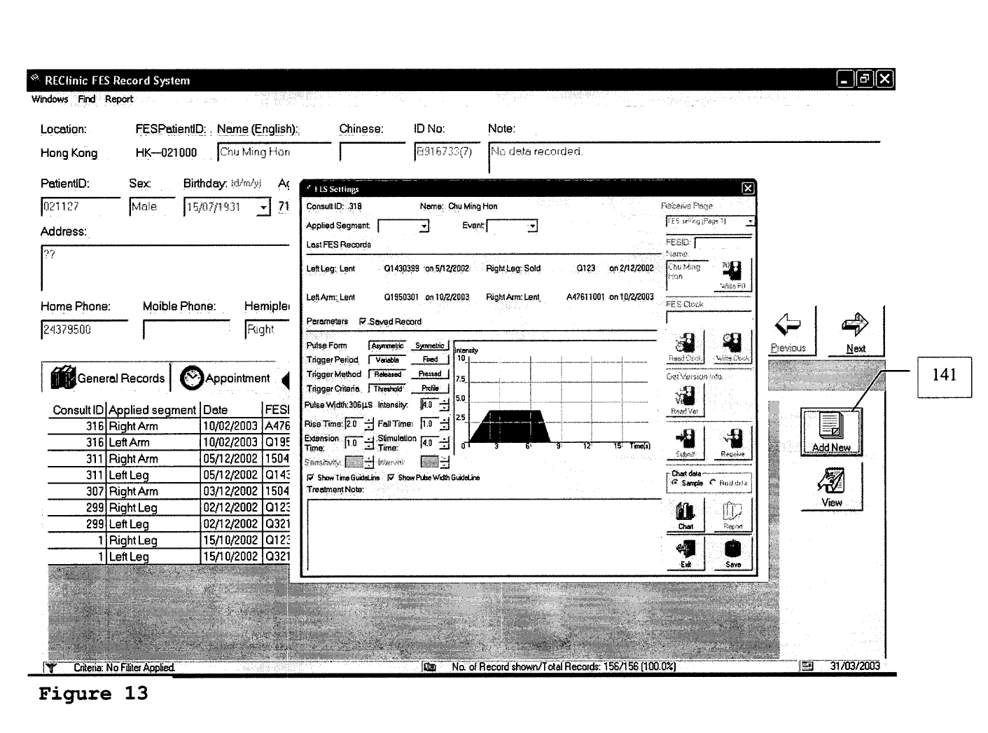

Referring to FIGS. 11 and 12, you can review past settings of the FES system by highlighting the date of visit and click “View” (142). To set new settings for FES System or to change settings, select “Add New” (141) and input the new settings so that you can retrieve it anytime as shown in FIG. 13.

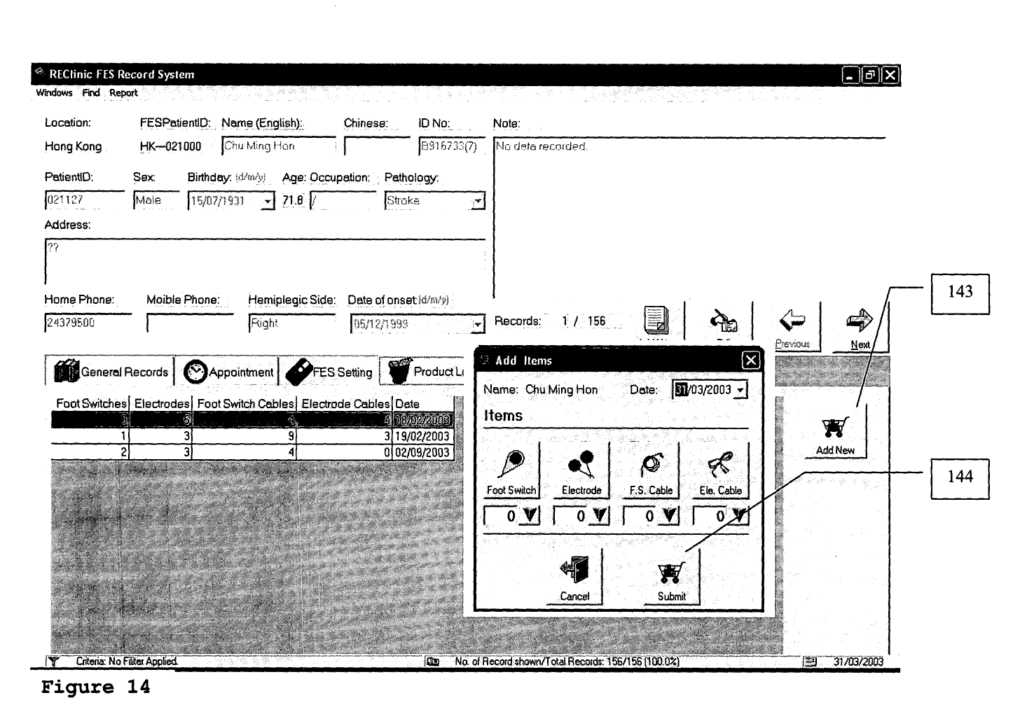

Referring to FIG. 14, physicians can record the number of accessories the patient has consumed by clicking “Add New” (143) or directly clicking on the specific icon to increase the item number. To decrease the number on a specific item, click the downward arrow under the corresponding icon directly and then click “Submit” (144).

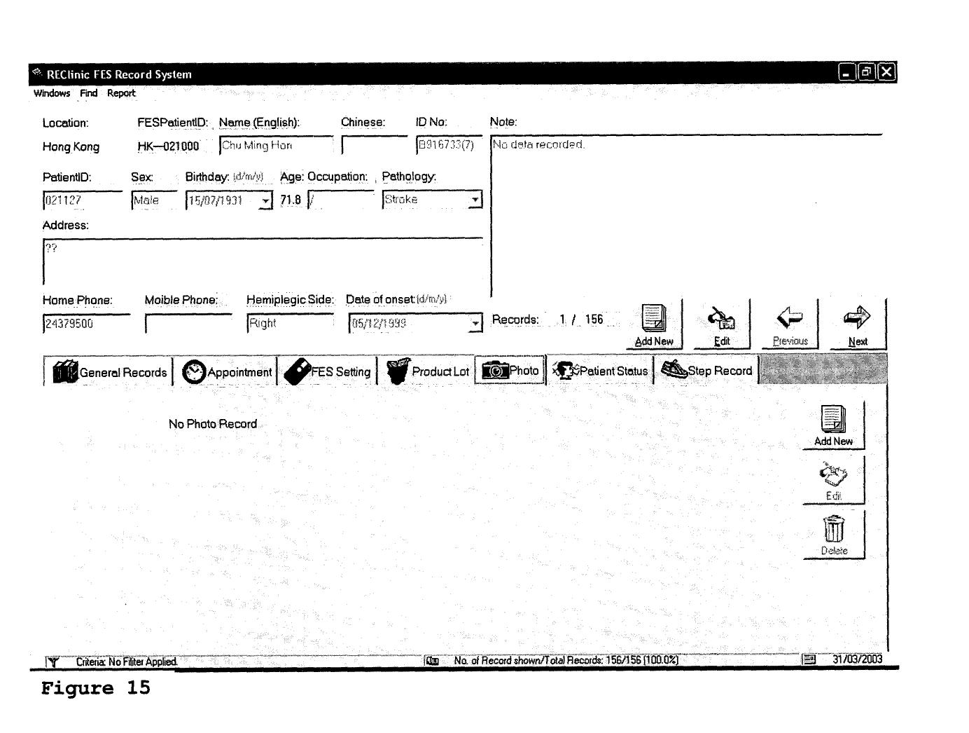

Referring to FIG. 15, physicians can upload photos to the FES Recording System. Photo can be, for example, electrode sites, skin condition before and after having electrode on patient's skin, toe clearance during walking with and without the application of FES on patient's affected foot.

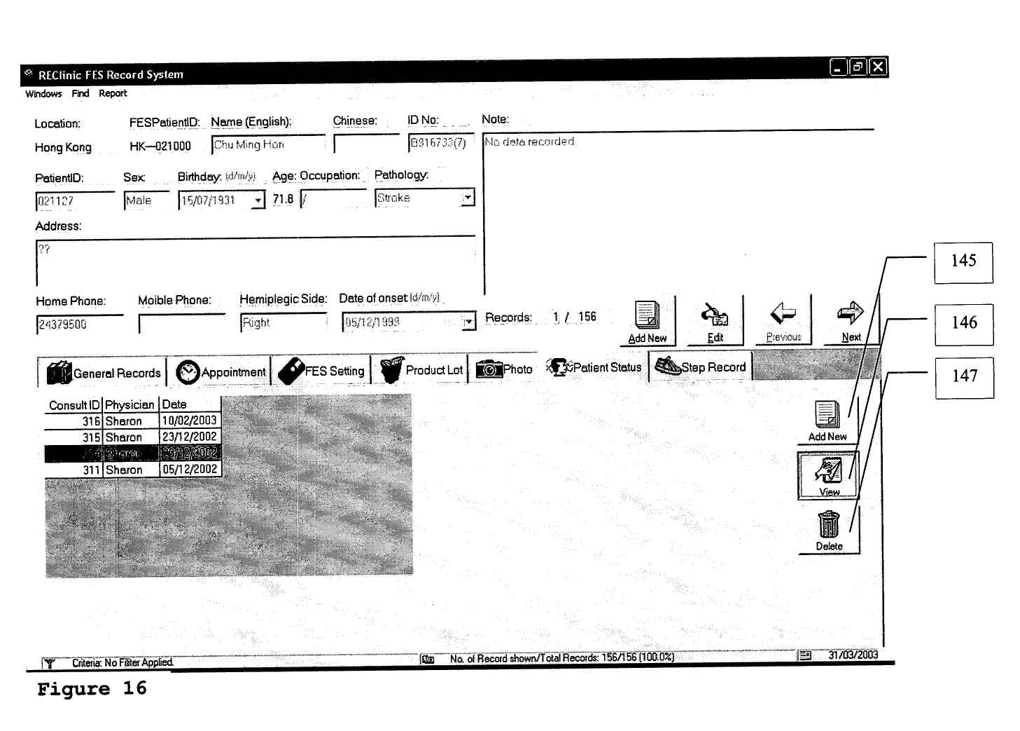

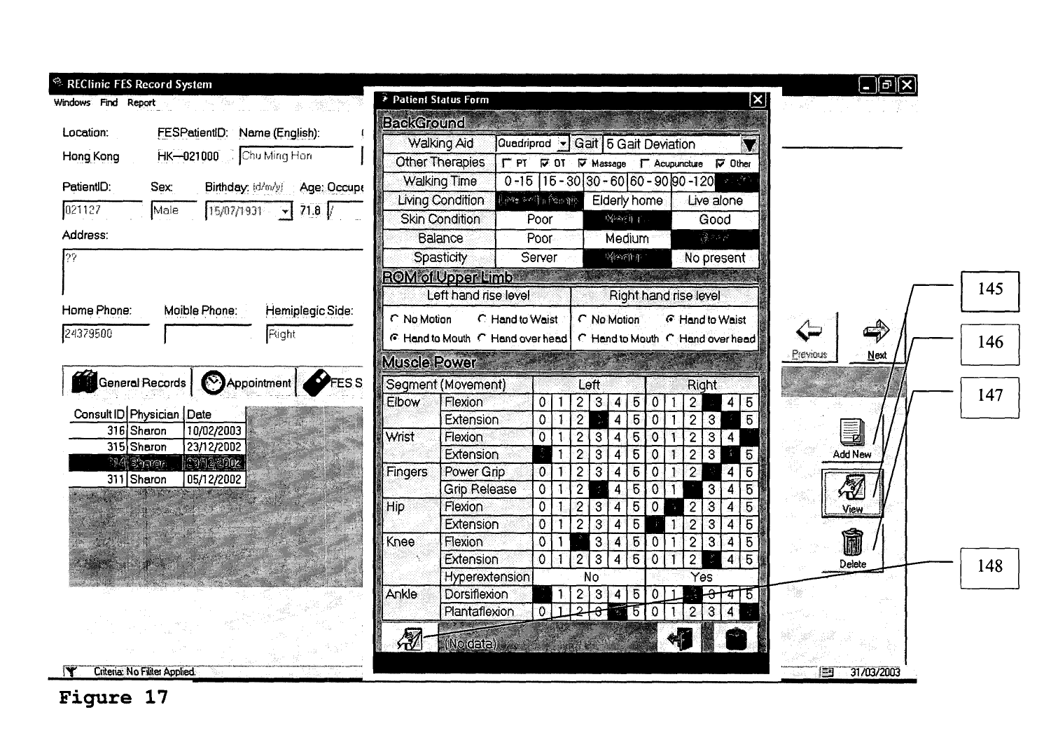

Referring to FIG. 16 and 17, physicians can update notes on a patient's physical condition during each visit. To review previous patient's status, select the date of visit you would like to review and click “View” (146). “Patient Status Form” will be shown as in FIG. 12. To update patient's physical status, click “Add New” (145), a blank Patient Status Form will then be shown. Select by directly clicking on the desired box. By clicking the icon (148), the physicians can type any other related information not included in the Form.

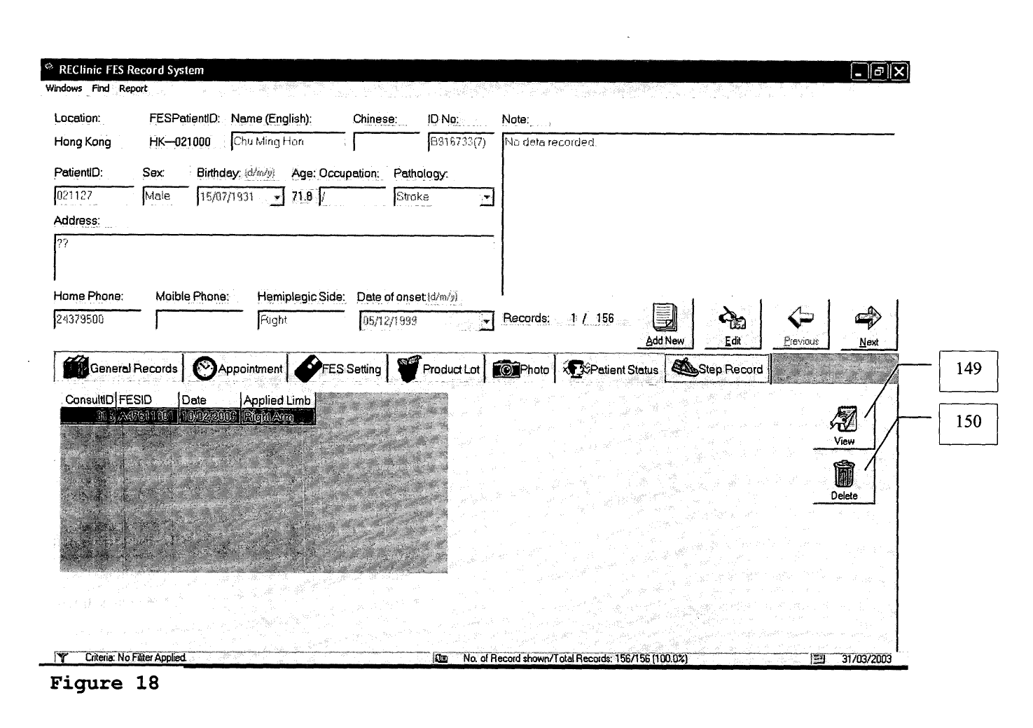

Referring to FIG. 18, physicians can view a patient's walking pattern at a particular stage of treatment by highlighting the desired date of visit and click “View” (149).

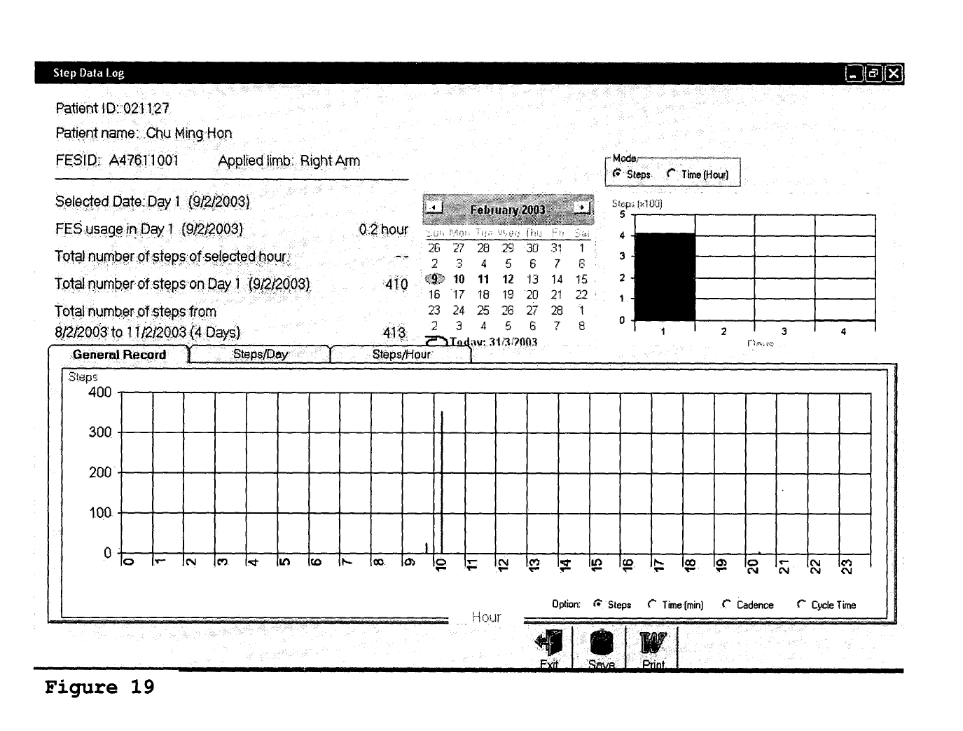

Referring to FIG. 19, 20 and 21, a patient's walking pattern using the FES System can be recorded. The number of steps and walking time in a selected hour, day or from a period of dates are shown in both numerical and graphic form. This information is logged by the wearable control module and downloaded to the database. Cadence is calculated by the software automatically. The physician can adjust the FES setting after monitoring patient's walking habit or after knowing patient's compliance.

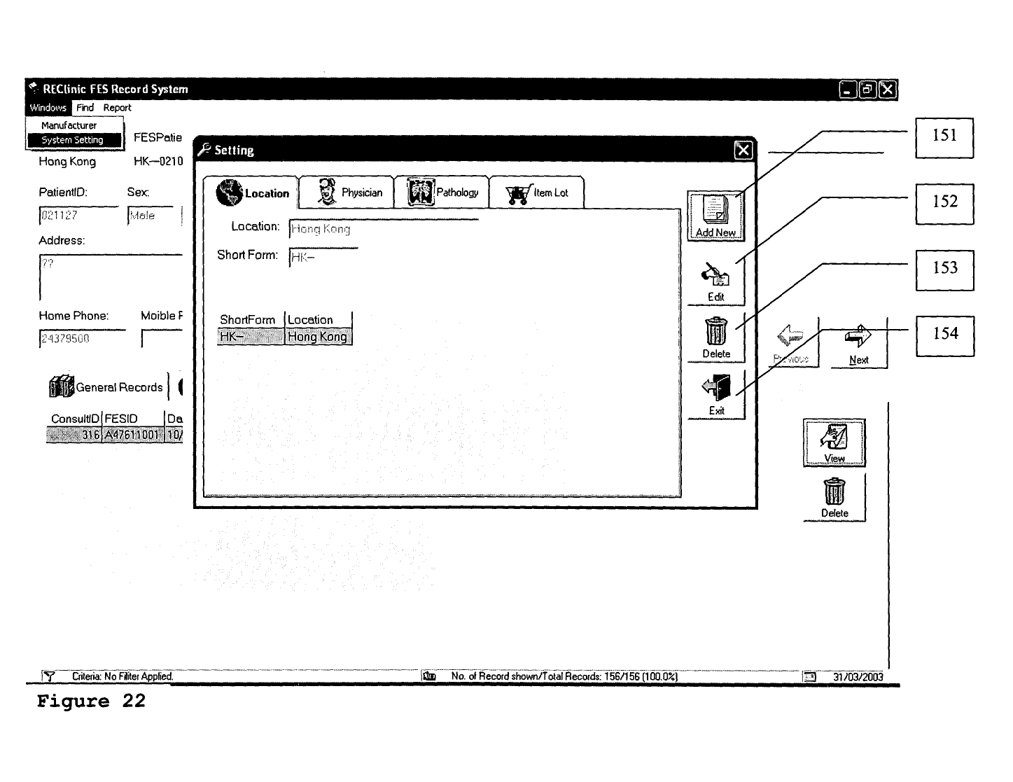

Referring to FIG. 22, physicians can add the name or location of their treatment facility by clicking “Add New” (151) and it will be showed on the front page of the system, as indicated by “Location” (104) in FIG. 1. To change or delete, physicians can click “Edit” (152) or “Delete” (153) respectively.

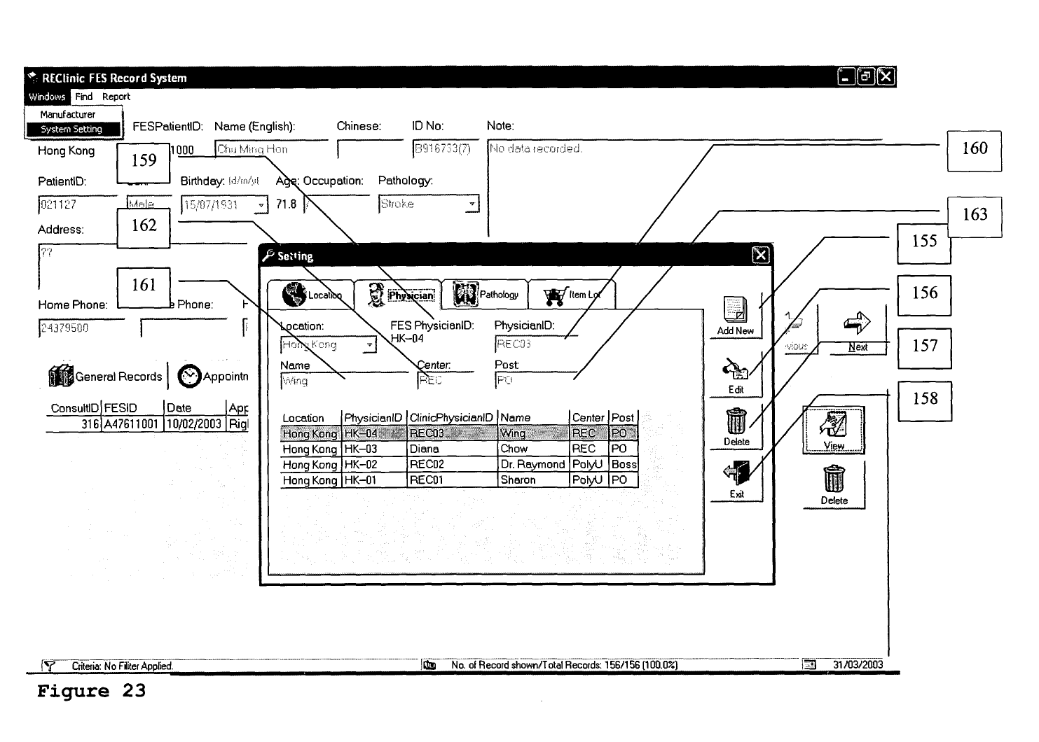

Referring to FIG. 23, physicians can add their name and their related information for booking appointment purpose by clicking “Add New” (155). To modify the information you can click “Edit” (156) or “Delete” (157) to delete it. FES Physician ID (159) will be created by the FES Recording System automatically.

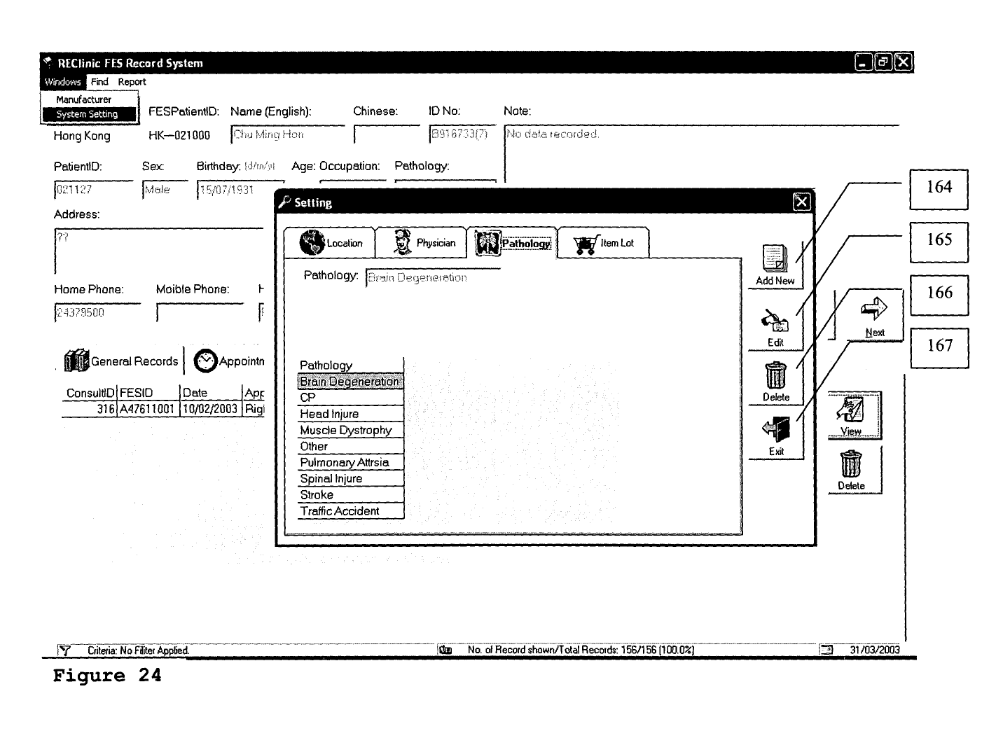

Referring to FIG. 24, you can add different diagnosis in the list by clicking “Add New” (164). To change or delete items in the list, you can click “Edit” (165) or “Delete” (166) respectively.

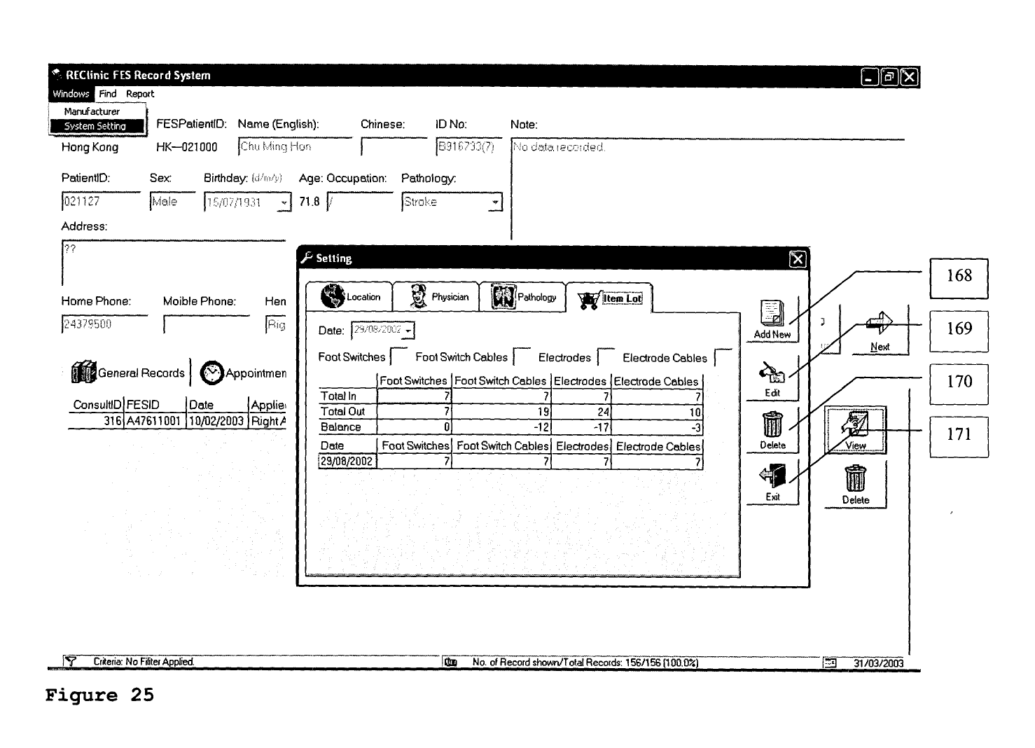

Referring to FIG. 25, the balance of accessories in your center can be recorded. You can add or delete number of items by clicking “Add New” (168) or “Edit” (169) respectively.

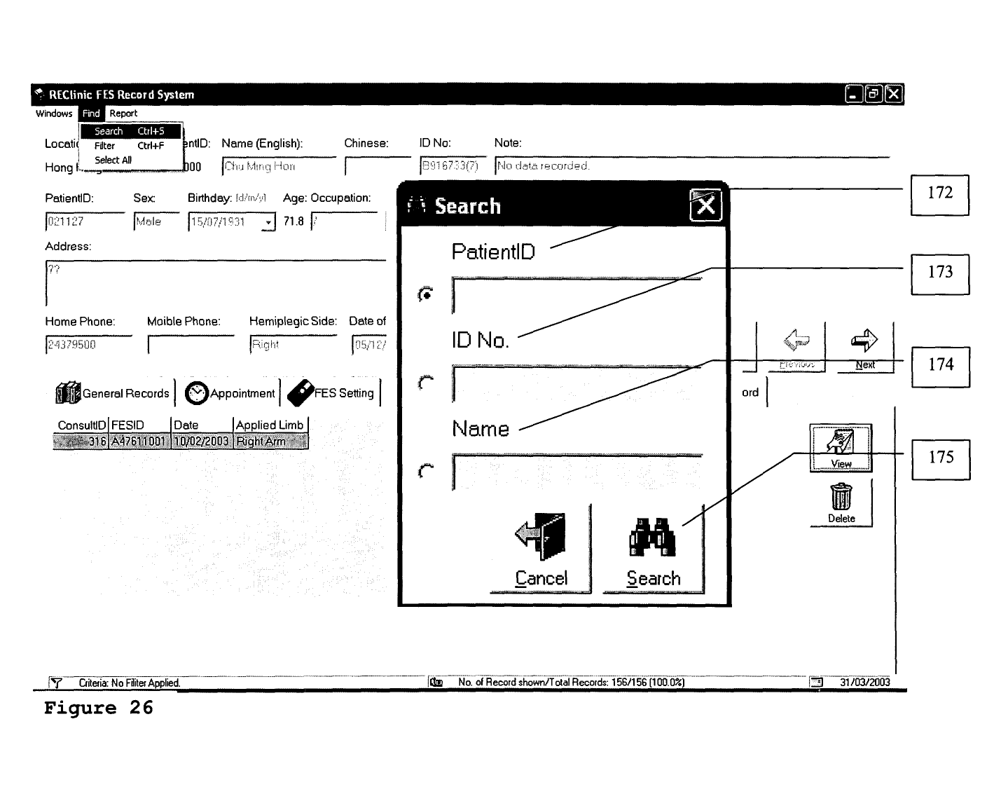

Referring to FIG. 26, you can search for a patient by using their PatientID (172), ID Card No. (173) or Name (174) and clicking “Search” (175).

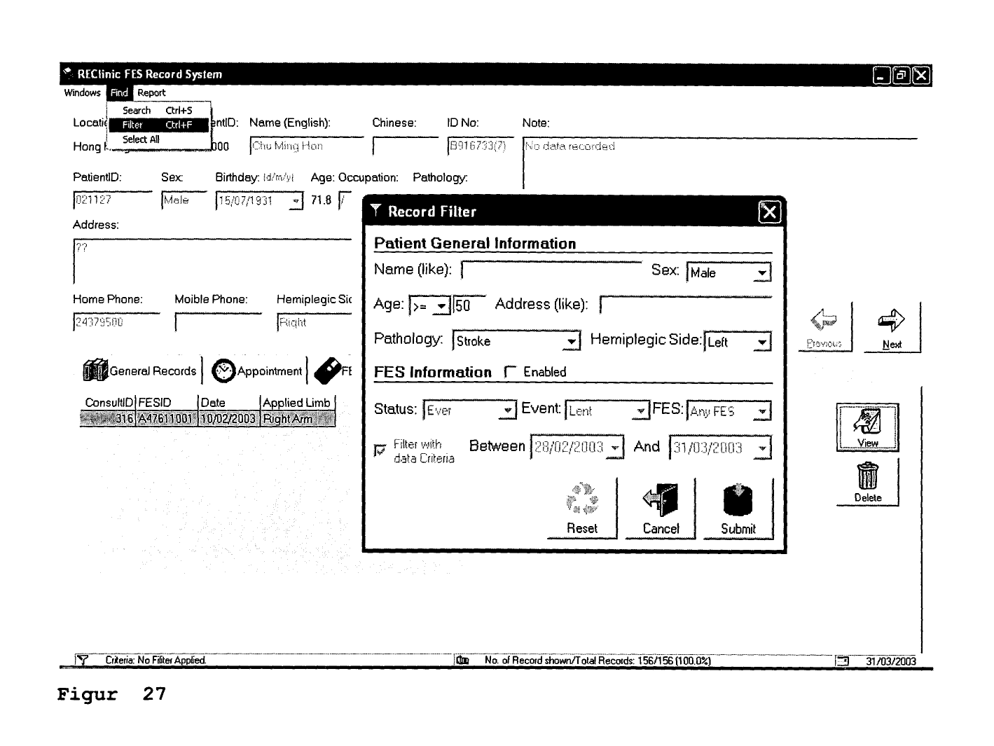

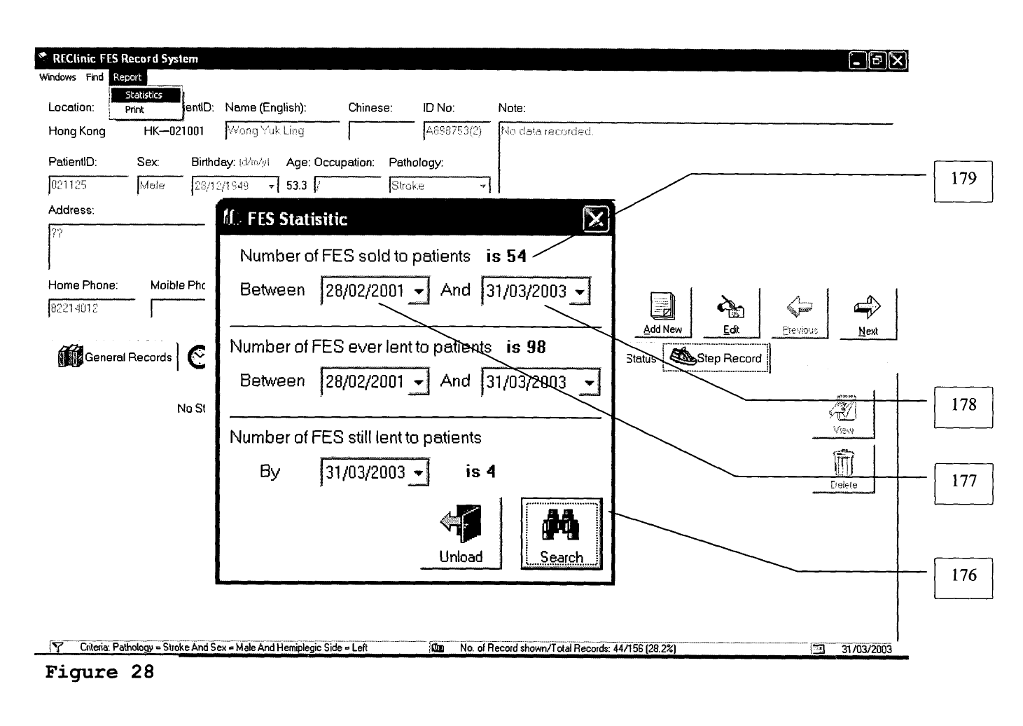

Referring to FIG. 27, “Filter” is used to select a specific group of patients with common criteria. For example the number of male patients aged 50 or more affected on his left side after a stroke. Referring to FIG. 28, “statistics” will tell you the status of the FES system. You can check the total number of FES systems sold or lent to patients in a certain period of time. You can also find out the number of FES system on loan to a patient. For example, to find out the number of FES Systems sold to patients from Feb. 28, 2001 to Mar. 31, 2003. Input “Feb. 28, 2001” in (177) and Mar. 31, 2003 in (178) and then click “Search” (176). The result shown is 54 units (179).

Where in the foregoing description reference has been made to integers or elements having known equivalents then such are included as if individually set forth herein.

Embodiments of the invention have been described, however it is understood that variations, improvements or modifications can take place without departure from the spirit of the invention or scope of the appended claims.

1. An electrical stimulation device comprising:
a sensor for detecting a movement event of a body part,
an electrode for making electrical contact with an area of the body part, and
a controller coupled to the sensor and electrode for receiving a sensor signal indicating the movement event, and for outputting to the electrode an output comprising a rise signal, a stimulation signal and a fall signal, and programmed to record a duration of use and a number of movement events during the duration of use.

2. An electrical stimulation device for controlling the movement of a body part comprising:
a sensor for detecting a movement event of a body part,
an electrode for making electrical contact with an area of the body part and for stimulating a muscle of the body part,
a housing to be worn by a user of the device, a receiver on the housing for receiving wireless signals from a remote unit, and
a controller provided in the housing and coupled to the receiver for receiving stimulation data from the remote unit and storing the stimulation data in a stimulation file, and coupled to the sensor for receiving a sensor signal indicating the movement event, and for generating a control signal using the stimulation file in response to the movement event, and for outputting the control signal to the electrode.

3. The device of claim 2 wherein the controller is also programmed for generating a log file storing a duration of use and a number of movement events during the duration.

4. The device of claim 2 wherein the stimulation data includes a stimulation intensity level, a rise time, a stimulation time, and a fall time.

5. The device of claim 4 wherein the stimulation data also includes a pulse form, a triggering period, a triggering method and triggering criteria.

6. The device of claim 2 further including a computer removably coupled to the controller for downloading the stimulation file and log file, and for updating the stimulation file, and programmed to store data from the stimulation and log files in a database, and for outputting for display the stimulation data and the duration of use and the number of movement events.

7. The device of claim 6 wherein the computer is a Personal Digital Assistant.

8. The device of claim 6 wherein the database also includes information about the user of the device, and the computer is programmed for accessing the database by a Windows™ graphical user interface.

9. The device of claim 2 wherein the remote unit is a handheld remote control unit.

10. The devise of claim 2 wherein the body part is a foot and the sensor is a heel switch.
